(19) United States
(12) Patent Application Publication
(10) Pub. No.: US 2024/0415663 A1
(43) Pub. Date: Dec. 19, 2024

(54) SACRAL FIXATION SYSTEM AND ASSEMBLY COMPRISING SUCH A SYSTEM

(71) Applicant: S.M.A.I.O., SAINT PRIEST (FR)

(72) Inventors: Poul Martin GEHRCHEN, Hellerup (DK); Clément SILVESTRE, Francheville (FR); Pierre ROUSSOULY, SAINT CYR AU MONT D'OR (FR); Sébastien CHAROSKY, Vieille Toulouse (FR); Jean-Charles ROUSSOULY, Poleymieux (FR)

(21) Appl. No.: 18/742,730

(22) Filed: Jun. 13, 2024

(30) Foreign Application Priority Data
Jun. 16, 2023 (FR) 2306212

(51) Int. Cl. A61F 2/30; A61B 17/00; A61B 17/56; A61B 17/70; A61B 17/80

(52) U.S. Cl. CPC A61F 2/30988; A61B 17/7059; A61B 17/8014; A61B 17/8057; A61B 2017/00477; A61B 2017/568; A61F 2002/30995

(57) ABSTRACT

A sacral fixation system, comprising a plate (10) comprising:
a proximal face (16) and a distal face (17), a plate plane extending between the distal and proximal faces,
a first housing (21) extending along a first axis (X21)
a second housing (22) extending along a second axis (X22),
a plate axis, the second axis forming a first angle with the plate plane, and the second axis and the plate axis forming a second angle comprised between 45 degrees and 110 degrees;
a second screw (12), received in the second housing to penetrate at least the vertebra S2 of the patient's sacrum;
a locking cap (13), which comprises a skirt (36), being received in the second housing by interposing the skirt between the second screw and the second housing.

### FIELD OF THE INVENTION

The present invention relates to a sacral fixation system and an assembly comprising such a fixation system.

### BACKGROUND OF THE INVENTION

For posterior arthrodesis fixtures of the spine, it is known to use a sacral fixation system, e.g. in the form of a plate that is attached to the sacrum of a patient, more particularly to the vertebrae S1 and S2, by means of screws.

The sacral fixation system in the form of a plate makes possible, on the one hand, a distal offset of the connection between the spinal rod of the arthrodesis fixture and the sacral fixation system, compared to the use of a single pedicle screw in the vertebra S1. As a result, the lumbar lordosis is concentrated close to the sacrum without the risk of conflict between anchors of the vertebra L5 and the sacrum.

On the other hand, when the posterior arthrodesis assemblies of the spine reach the pelvis, the sacral fixation system represents the base of the fixture. Therefore, the sacral fixation system requires robustness providing thereto, in particular, a high resistance to a pull-out force to which same is subjected when assembled on the sacrum. The sacral fixation system in the form of a plate, benefiting from two screws inserted into the vertebrae S1 and S2, respectively, of the sacrum, has improved robustness compared to a pedicle screw inserted into the sacrum.

Such systems are satisfactory overall, but the strength of the fastening screws of the fixture may be insufficient over time, which weakens the fixture and limits the lifetime thereof.

### SUMMARY OF THE INVENTION

The goal of the present invention is to propose an improved sacral fixation system having improved robustness and lifetime.

To this end, the subject matter of the invention relates to a sacral fixation system, comprising a plate comprising a proximal face and a distal face, opposite the proximal face, a plate plane extending between the distal and proximal faces, a first housing extending along a first axis connecting the distal and proximal faces to each other, the first housing including both a first hole, which opens onto the distal face, and a first bottom, wherein the first hole is pierced and which connects the first hole and the proximal face, a second housing extending along a second axis connecting the distal and proximal faces to each other, the second housing including both a second hole, which opens onto the distal face, a tapped surface, which opens onto the proximal face, and a second bottom, which has a concave shape, wherein the second hole is drilled and which connects the second hole and the tapped surface, and a plate axis belonging to the plate plane and intersecting the first and second axes, the second axis forming, in projection onto a plane perpendicular to the plate axis, a first angle comprised between 30 and 60 degrees with the plate plane, and the second axis and the plate axis forming therebetween a second angle comprised between 45 degrees and 110 degrees. The system further comprises a first screw, which, when the system is in an assembled configuration, is received in the first housing, extending into the first hole and emerging from the distal face so as to be able to penetrate the vertebra S1 of the patient's sacrum and a second screw, which comprises a threaded rod and a head, the head being substantially spherical matching the second bottom, the second screw, when the system is in the assembled configuration, being received in the second housing so that the head rests on the second bottom and the rod extends into the second hole and emerges from the distal face so as to be able to penetrate at least into the vertebra S2 of the patient's sacrum. The system further comprises a locking cap, which includes a skirt having an outer face that is threaded so as to match the threaded surface and an inner face that matches the head of the second screw, and which, when the system is in the assembled configuration, is received in the second housing by interposing the skirt radially between the head of the second screw and the threaded surface, so that the head of the second screw is received in the skirt and is pressed against the inner face and a connecting device, which, when the system is in the assembled configuration, is arranged on the proximal face between the first and second housings and connects the plate to a spinal rod.

One of the ideas underlying the invention is to make the angle of the axis of the second housing, which is fixed for a given plate, take a value over a wide range of predetermined values, namely the range extending from 45 degrees to 110 degrees. Thereby, the fixation system makes it possible to choose the most suitable position for the second screw when same is in the assembled configuration, in order to ensure that the screw penetrates the vertebra S2 of the patient's sacrum without damaging other organs, such as nerves or blood vessels.

In addition, when the fixation system is in the assembled configuration and implanted in the patient, same is subjected to a pulling force which is likely to cause the pulling of the system out of the sacrum, a phenomenon which should be limited. By means of the angulation of the second housing, the invention serves to obtain a better mechanical strength of the fixing system. In fact, the second screw penetrates, according to the angulation imposed by the second housing, at least into the vertebra S2 of the patient, and can advantageously extend beyond, if need be. Thereby, the invention makes it possible to adapt the orientation of the second housing, and hence of the second screw, to the particular context of each patient in order to optimize the mechanical strength of the second screw in each case and prevent damage to other organs.

The robustness of the sacral fixation system is also ensured over time due to the locking cap. The skirt of the locking plug is interposed radially between the tapped surface of the second housing and the head of the second screw, and presses against the head of the second screw, which causes the head of the second screw to bear against the bottom of the second housing. The two supports generate a friction force sufficient to completely lock the second screw. More particularly, the second screw cannot pivot in the second housing. Same can thereby neither unscrew and come out of as such from the plate, nor pivot in the housing thereof and weaken the system, by modifying the relative orientation of the plate and of the second screw. The locking cap thereby prevents the second screw from moving when the system is in the assembled configuration. The invention is thus robust and has an optimized resistance, in particular to the tear-off force, which leads to good mechanical strength in the bone and hence a long lifetime.

The system can comprise one or a plurality of the following features, taken individually or according to any technically possible combination:
The second angle is between 70 degrees and 110 degrees, preferably comprised between 85 and 95 degrees, and the second screw is dimensioned so that, in the assembled configuration, same passes through the vertebra S2 and reaches the iliac bone of the patient.
The plate further comprises a recess and the connection device comprises:
   an threaded extension, fixedly connected to the recess of the plate, the extension extending along an axis of extension;
   a connector, comprising a proximal dome cap and a distal dome cap, the distal dome cap matching the recess, each dome cap comprising:
      an orifice, with a diameter greater than a diameter of the extension, the orifices of the proximal and distal dome caps being coaxial and centered on a connector axis,
      a bearing surface, the bearing surfaces facing each other along the connector axis;
      each dome cap being movable along the connector axis in order to bring the bearing surfaces into contact with each other along the connector axis and to tighten the connector around the vertebral rod,
   a nut, comprising a tightening portion, the tightening portion comprising a distal surface matching the proximal dome cap,
and wherein, when the system is in the assembled configuration:
   the connector being fitted onto the extension through the holes in the distal and proximal dome caps; and
   the nut being screwed onto the extension, the distal surface bears against the proximal dome cap, the nut holding the distal dome cap bearing against the recess and keeping the bearing surfaces in contact along the connector axis; and
   the connector being tightened around the vertebral rod.
The proximal and distal dome caps are hemispherical and when the system is in the assembled configuration, the lower and upper dome caps are co-radial.
The nut further comprises a screwing portion, supported by the tightening portion, configured to detach from the tightening portion when a torque equal to a maximum screwing torque is applied to the screwing portion.
The extension comprises a line of least resistance making same breakable when a bending or torsional torque equal to a bending or torsional breaking torque is applied to the extension.
The connection device comprises:
   a pin, fixedly supported by the plate;
   a tulip-shaped piece extending along an axis of a tulip-shaped piece, the pin and the tulip-shaped piece forming therebetween, a ball-and-socket joint, the tulip-shaped piece comprising a threaded through opening;
   a disc, comprising a distal surface matching the pin and a proximal surface matching the vertebral rod;
   a cap, suitable for being screwed into the opening of the tulip-shaped piece, when the system is in the assembled configuration:
   the disc being placed in the opening of the tulip-shaped piece, the distal surface of the disc bearing against the pin;
   the vertebral rod passing through the opening of the tulip-shaped piece, bearing against the proximal surface of the disc;
      the cap is screwed into the opening of the tulip-shaped piece and holds the vertebral rod, the disc and the pin bearing against each other.
The plate axis passes through the pin.
The pin is offset from the plate axis along a direction perpendicular to the plate axis.

A further subject matter of the invention is a sacral fixation assembly comprising the system described hereinabove and a drill guide comprising:
a distal surface morpho-adapted to a posterior region of the patient's sacrum;
a first guide hole centered along a first guide axis, and
a second guide hole centered along a second guide axis, the first guide hole and the second guide hole being oriented identically to the first and second screws when the system is in the assembled configuration.

### BRIEF DESCRIPTION OF THE DRAWINGS

The invention will be clearer upon reading the following description, given only as an example, but not limited to, and making reference to the figures wherein:

FIG. 1 is a view of a sacral fixation system implanted in a patient according to a first embodiment of the invention;

FIG. 2 is a perspective view of a plate of the sacral fixation system according to the embodiment;

FIG. 3 shows, on two inserts a) and b), elevation views of the plate shown in FIG. 2, associated with the first and second fastening screws, insert a) being an elevation view along the direction of arrow IIIa in FIG. 2 and insert b) being an elevation view along the direction of arrow IIIb on insert a);

FIG. 4 is a section along the line IV-IV shown in FIG. 1;

FIG. 5 shows, on two inserts a) and b), elevation views of the plate associated with the first and second fastening screws shown in FIG. 3, the plate and the fastening screws being schematically associated with bone material, the insert a) being a view corresponding to insert a) of FIG. 3, and insert b) being an elevation view along the direction of arrow Vb on insert (a);

FIG. 6 is a schematic section along the line VI-VI shown in FIG. 3; and

FIG. 7 shows, on two inserts a) and b), schematic sections along line VII-VII of insert b) of FIG. 3, where the plate is associated with a connection device, insert a) being a section where the connecting device is in free configuration and insert b) being a section where the connecting device is in tight configuration;

FIG. 8 shows, on two inserts a) and b), schematic sections where the travel of the connection device is shown, insert a) being a section corresponding to insert a) of FIG. 7 and insert b) being a schematic section along plane VI-VI of insert b) of FIG. 3, the plate being associated with the connection device;

FIG. 9 is a view similar to the view shown in FIG. 4, according to a second embodiment of the invention;

FIG. 10 shows two inserts a) and b) similar to the inserts a) and b), respectively, of FIG. 5, the plate being according to the second embodiment of the invention;

FIG. 11 is a schematic perspective view of a vertebral plate and of a connector according to a third embodiment of the invention;

FIG. 12 is a section along plane XII-XII of the plate shown in FIG. 11, where the travel of the connection device is shown;

FIG. 13 is a view similar to the view shown in FIG. 11, the plate being according to a fourth embodiment of the invention; and

FIG. 14 shows, on an insert a), an elevation view of a drill guide and, on an insert b), an elevation view of the drill guide along the direction of arrow XIVb of insert a).

### DETAILED DESCRIPTION

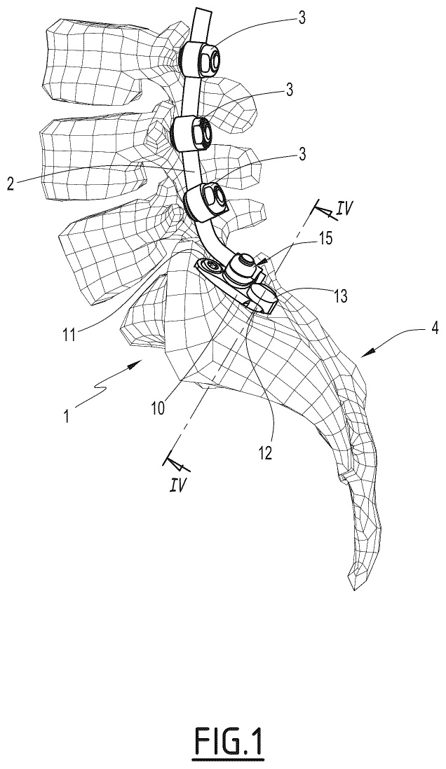

FIG. 1 shows a sacral fixation system 1, also called system 1. System 1 belonging to an arthrodesis fixture, also comprising a vertebral rod 2 and a plurality of pedicle screws 3. System 1 is shown here in a configuration assembled and fastened to the sacrum 4 of a patient, more particularly to the vertebrae S1 and S2 of the sacrum 4 of the patient, as explained in greater detail thereafter.

The arthrodesis fixture relates herein to one of two sides among a left side and a right side of the spine of the patient. In FIG. 1, the arthrodesis fixture shown is the left side. Where appropriate, a second fixture is fastened to the right side, which comprises arrangements that are overall symmetrical to same of the arthrodesis fixture on the left side with respect to a sagittal plane of the patient. More particularly, the system of the arthrodesis fixture located on the right side is overall symmetrical to system 1 with respect to the sagittal plane of the patient.

System 1 comprises a plate 10, a first fastening screw 11, a second fastening screw 12, a locking cap 13 and a connection device 15.

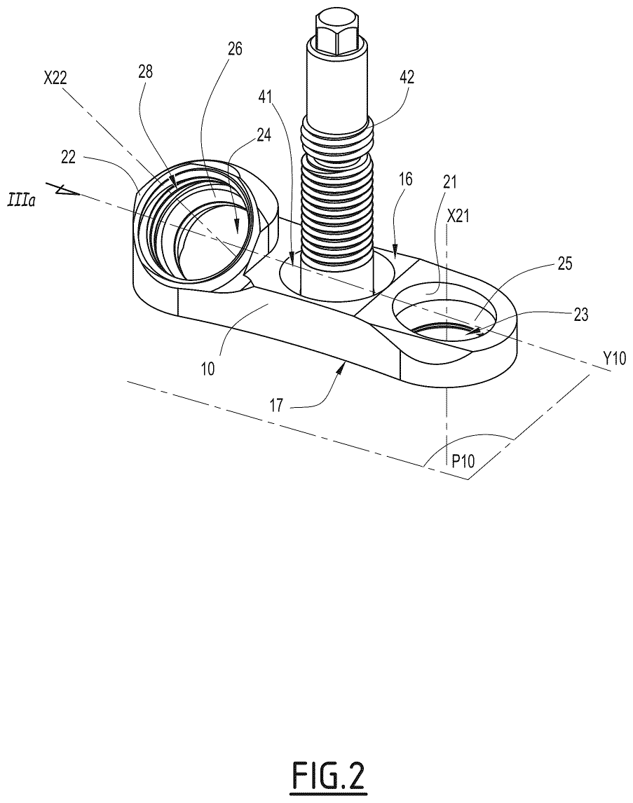

As can be seen clearly in FIG. 2, the plate 10 comprises a proximal face 16 and a distal face 17, opposite the proximal face 16, in the sense that the proximal face 16 and the distal face 17 are separated from each other by the thickness of the plate 10, i.e. the smallest of the three dimensions of the plate 10. P10 denotes a geometric plate plane which extends between the proximal face 16 and the distal face 17.

In the embodiment illustrated in FIGS. 1 to 8, the distal face 17 is flat, i.e. parallel to the plane P10. Alternatively (not shown), the distal face is curved, more particularly concave, in order to better fit in with a kyphotic curvature of the sacrum 4.

The plate 10 comprises two distinct housings, namely a first housing 21 and a second housing 22. The first housing 21 connects the proximal 16 and distal 17 faces to each other, extending along a first axis X21. The second housing 22 connects the proximal 16 and distal 17 faces to each other, extending along a second axis X22. The first and second axes X21 and X22 define a plate axis Y10, by the intersection thereof with the plate plane P10. In other words, the plate axis Y10 belongs to the plate plane P10 and is secant with the first and second axes X21 and X22.

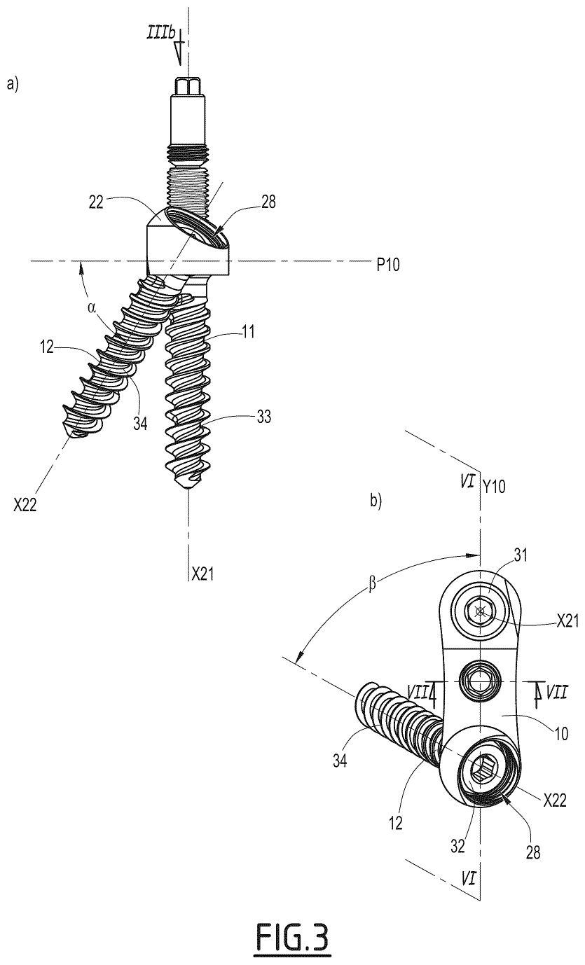

As can be seen clearly in FIG. 2 and in inserts a) and b) of FIG. 3, the first axis X21 is herein perpendicular to the plate plane P10.

Also, as can be seen clearly in FIG. 2 and in inserts a) and b) of FIG. 3, the second axis X22 is also transverse to the plate plane P10, without, however, being parallel to the first axis X21.

More precisely, as shown in insert a) of FIG. 3, the second axis X22 forms, in projection onto a geometric plane perpendicular to the plate axis Y10, a first angle α with the plate plane P10, the first angle α being comprised between 30 and 60 degrees, preferentially equal to 45 degrees, the first angle α being measured on the side of the plate 10, intended to be turned in the opposite direction from the sagittal plane of the patient.

In addition, as shown in insert b) of FIG. 3, the second axis X22 forms a second angle β with the plate axis Y10, the second angle β being comprised between 70 and 110 degrees in the embodiment considered in FIGS. 1 to 8, the second angle β being measured on the side of the plate 10 intended to be turned upwards with respect to the patient. In other words, the second angle β is formed in a geometric plane formed by the plate axis Y10 and the second axis X22. For reasons which will become apparent further down, the second angle β is preferentially comprised between 85 and 95 degrees, or even equal to 90 degrees.

As can be seen clearly in FIG. 2, the first housing 21 comprises a first hole 23, which opens onto the distal face 17, and a first bottom 25 which connects the first hole 23 and the proximal face 16. The first hole 23 is drilled in the first bottom 25, being centered herein on the first axis X21. The first hole 23 is advantageously frustoconical, so that a diameter of the first hole 23 measured at the junction between the first hole 23 and the distal face 17 is greater than a diameter measured at the junction between the first hole 23 and the first bottom 25. The first bottom 25 has herein a substantially hemispherical shape, i.e. has the shape of a portion of a sphere centered on the first axis X21.

The second housing 22 comprises at the same time a second hole 24, which opens onto the distal face 17, a tapped surface 28, which opens onto the proximal face 16, and a second bottom 26 which connects the second hole 24 and the tapped surface 28 to each other. The second hole 24 is drilled in the second bottom 26 and is centered herein on the second axis X22. The second hole 24 is advantageously frustoconical, so that a diameter of the second hole 24 measured at the junction between the second hole 24 and the distal face 17 is greater than a diameter measured at the junction between the second hole 24 and the second bottom 26. The second bottom 26 has a substantially concave shape, more particularly hemispherical, i.e. has the shape of a portion of a sphere centered on the second axis X22. The second bottom 26 advantageously has a diameter equal to a diameter of the first bottom 25. The tapped surface 28 is centered on the second axis X22 and extends between the second bottom 26 and the proximal face 16.

For reasons which will become apparent later, the plate 10 comprises a recess 30, located more precisely on the proximal face 16. The recess 30 is advantageously hemispherical and centered on a recess axis X30 perpendicular to the plate plane P10. The recess axis X30 is located between the first and second housings 11 and 12, and advantageously the recess axis X30 intersects the plate axis Y10.

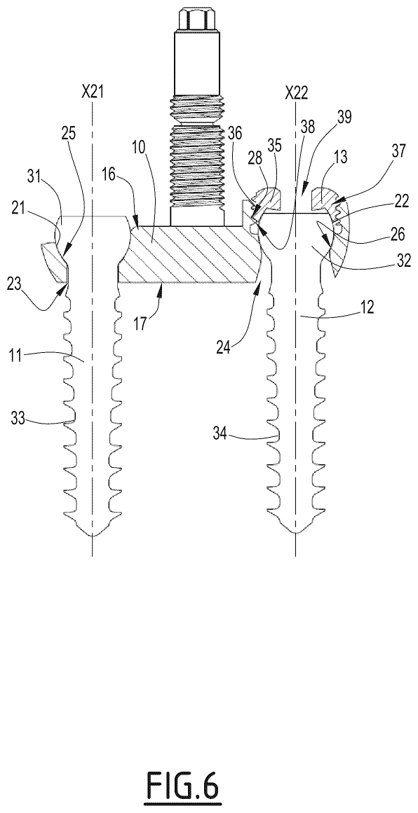

As can be seen clearly in FIG. 6, the first screw 11 comprises a substantially spherical head 31, with a shape matching the first base 25, and a threaded rod 33. The second screw 12 also comprises a substantially spherical head 32, with a shape matching the second bottom 26, and a threaded rod 34. The first and second screws 11 and 12 are advantageously identical to each other.

When the system 1 is in the assembled configuration, as shown in FIGS. 3 to 6, the first screw 11 is received in the first housing 21. The head 31 of the first screw 11 bears against the first bottom 25, and the rod 33 extends into the first hole 23 and passes through same, thereby protruding from the distal face 17 to penetrate into the vertebra S1 of the sacrum 4 and thereby fasten the plate 10 to the sacrum 4 of the patient. As mentioned hereinabove, the head 31 of the first screw 11 and the first bottom 25 have a shape matching that the head 31 and the first bottom 25 are pressed against each other, forming a sphere-sphere contact. As illustrated schematically in FIGS. 3, 5 and 6, in the assembled configuration, the first screw 11 extends lengthwise substantially along the first axis X21, i.e. the central longitudinal axis thereof coincides with the first axis X21, or else forms with the latter, an angle of only a few degrees, typically less than 15 degrees.

According to an alternative embodiment (not shown), the first screw 11 forms an angle greater than 15 degrees with the first axis X21.

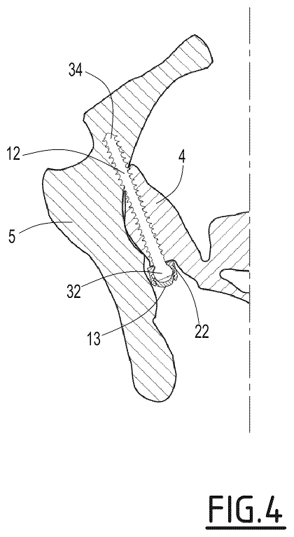

When the system 1 is in the assembled configuration, as shown in FIGS. 3 to 6, the second screw 12 is received in the second housing 22. The head 32 of the second screw 12 bears against the second bottom 26, in a sphere-sphere contact, and the rod 34 extends into the second hole 24 and passes through same, thereby protruding from the distal face 17 to penetrate into the vertebra S2 of the patient's sacrum 4, as can be seen in FIG. 4, and thereby fasten the plate 10 to the patient's sacrum 4, in addition to the first screw 11. As mentioned hereinabove, and in a manner similar to the first screw 11, the head 32 and the second bottom 26 have a matching shape, so that the head 32 of the second screw 12 and the second bottom 26 are pressed against each other, forming a sphere-sphere contact.

In practice, in the assembled configuration, the second screw 12 extends lengthwise substantially along the second axis X22, i.e. the central longitudinal axis thereof coincides with the second axis X22, as illustrated schematically in FIGS. 3, 5 and 6, or else forms with the latter an angle of only a few degrees, typically less than 15 degrees. In any case, the angulation of the second axis X22, as defined hereinabove through the first and second angles α and B, allows, in the assembled configuration, the second screw 12 to penetrate the vertebra S2 until same can, since the second screw 12 is dimensioned in length in an ad hoc manner, pass through the vertebra S2 and penetrate into the iliac bone 5 of the patient, as illustrated schematically in FIG. 4. As a preferential example, the second screw 12 has for this purpose, a longitudinal dimension comprised between 40 mm and 120 mm, preferentially between 50 mm and 110 mm. The fixation both in the vertebra S2 of the sacrum 4 and in the iliac bone 5 of the patient optimizes the mechanical strength of the system 1.

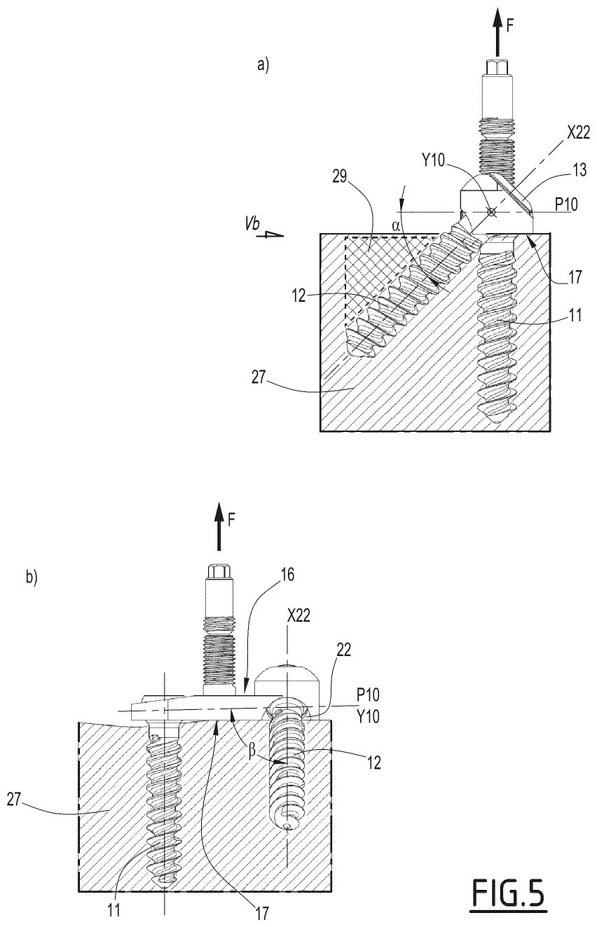

It should be noted that, in service, i.e. when the patient on whom the system 1 in the assembled configuration has been implanted, stresses his/her vertebral column in motion, the system 1 is typically subjected to a pulling force F which, as illustrated in the inserts a) and b) of FIG. 5, is exerted perpendicularly to the plate plane P10, being oriented from the distal face 17 toward the proximal face 16. The second screw 12 effectively opposes the force F due to the alignment thereof substantially with the second axis X22.

To better illustrate such phenomenon, inserts a) and b) of FIG. 5 show the first and second screws 11 and 12 penetrating into the bone material 27, the bone material 27 being represented schematically by a hatched area representing in an undifferentiated manner, the sacrum 4 and the iliac bone 5 of the patient. The bone 27 comprises a bone region 29, shown by dotted lines and doubly hatched. The bone region 29 is located between the second screw 12 and the plate plane P10 and limits the movement of the second screw 12 due to the pulling force F, which prevents the system 1 from being torn off. The existence of the bone region 29 is due to the angulation of X22, and in particular the value of the first angle α. It is thus particularly advantageous to have the first angle α comprised between 30 and 60 degrees in order to maximize a volume of the bone region 29.

The locking cap 13 makes it possible to prevent the second screw S2 from moving relative to the plate 10, in particular from unscrewing, under the action of the force F and/or of other forces. As can be seen clearly in FIGS. 4 and 6, the locking cap 13 comprises for this purpose, a skirt 36 centered on a geometric axis which, in the assembled configuration of the system 1, is aligned with the second axis X22. By definition, the skirt 36 comprises two faces separated from each other by the thickness thereof, namely an outer face 37 and an inner face 38. The outer face 37 is threaded to match the tapped surface 28, thereby enabling the locking cap 13 to be housed and fastened inside the second housing 22 by screwing between the tapped surface 28 and the thread of the outer face 37 when the system 1 is in the assembled configuration. The inner face 38 matches the head 32 of the second screw 12, thereby being hemispherical, thus enabling the head 32 to be received inside the skirt 36 and to be pressed, in a matching way, against the inner face 38.

In the assembled configuration of the system 1, the skirt 36 is interposed, radially with respect to the second axis X22, between the head 32 of the second screw 12 and the tapped surface 28 of the second housing 22. Since the locking cap 13 is screwed into the second housing 22, same presses against the head 32 of the second screw 12, in order to press the inner face 38 of the skirt 36 against the head 32, and thereby press the head 32 against the second bottom 26. Thereby, the second screw 12 is held immobile in the second housing 22, including during pivoting, more particularly about the second axis X22.

At the axial end of the skirt 36, which is turned axially in the opposite direction from the second screw 12 in the assembled configuration of the system 1, the locking cap 13 advantageously includes a head 35. An indentation 39 is made in a central part of the head 35, the indentation 39 allowing the locking cap 13 to be screwed on, e.g. using a screwing wrench. The skirt 36 is fixedly supported by the head 35, extending from a peripheral part of the head 35.

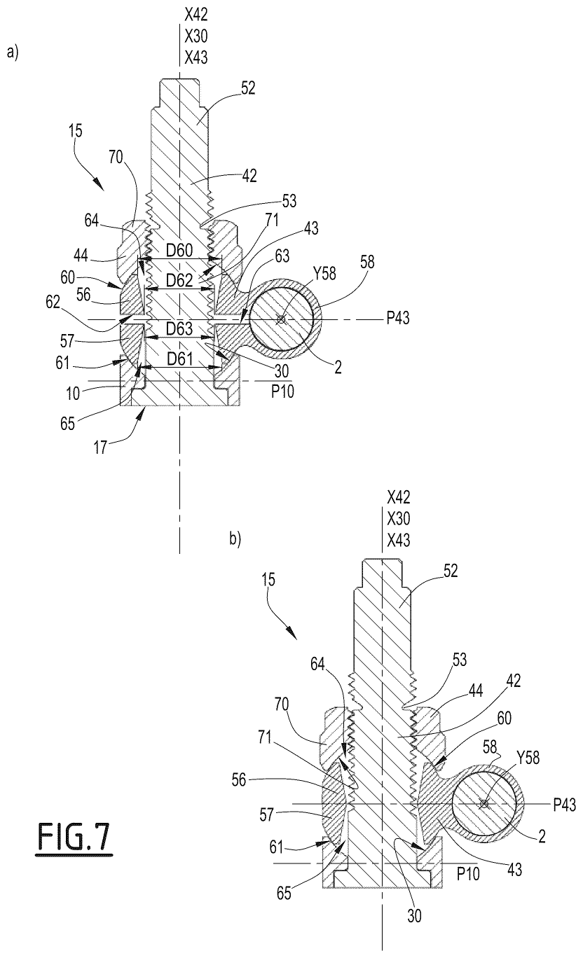

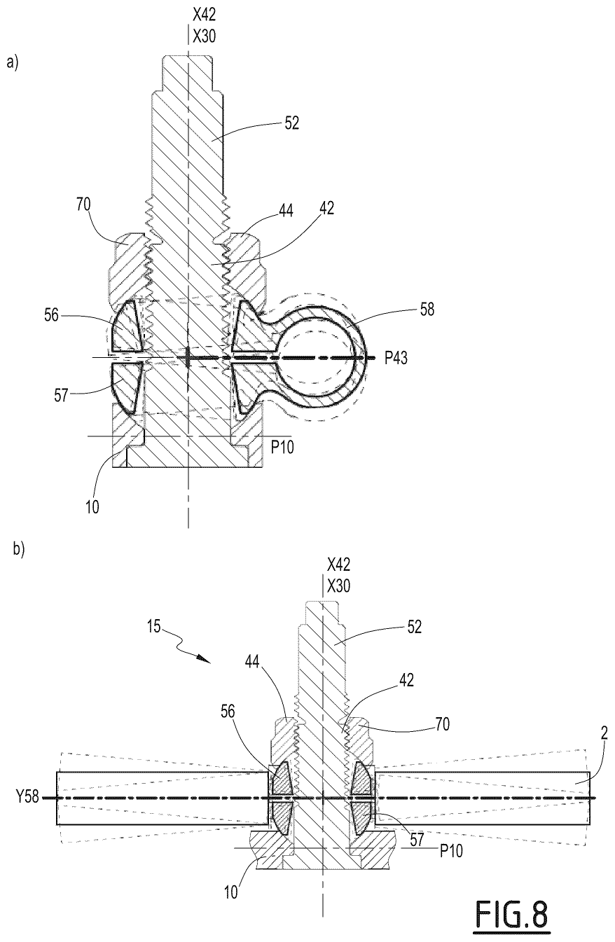

As can be seen clearly in inserts a) and b) of FIGS. 7 and 8, the connection device comprises an extension 42, a connector 43 and a nut 44.

The extension 42 extends along an extension axis X42, being fastened to the plate at the recess 30 in such a way that the extension axis X42 and the recess axis X30 coincide. As shown in inserts a) and b) of FIGS. 7 and 8, the extension 42 is formed by a screw shank passing through the plate 10 along the recess axis X30, the screw shank belonging to a screw which is welded to the plate 10. The extension 42 is at least partially threaded between the proximal and distal ends thereof, a proximal end portion 52 of the extension 42 advantageously not being threaded herein.

The extension 42 advantageously has a line of least resistance 53 designed to break when a bending or torsional torque equal to a bending or torsional breaking torque is applied to the proximal end of the extension 42. The above serves, in particular, to limit the size of the extension 42 once the system 1 has been assembled.

The connector 43 extends along a connector axis X43 and comprises a proximal dome cap 56, a distal dome cap 57 and a ring 58. The proximal 56 and distal 57 caps are located on either side of a median plane P43 and are centered on the connector axis X43.

The proximal dome cap 56 comprises a proximal surface 60 which has a hemispherical shape. The proximal dome cap 56 further comprises a bearing surface 62, which is a flat surface. The bearing surface 62 is arranged between the proximal surface 60 and the distal dome cap 57 along the connector axis X43.

The proximal dome cap 56 also comprises an orifice 64. The orifice 64 passes through the proximal dome cap 56 along the connector axis X43 and connects the proximal surface 60 of the proximal dome cap 56 to the bearing surface 62. A diameter of the orifice 64 is greater than or equal to the diameter of the extension 42. Advantageously, and as shown in the inserts of FIGS. 7 and 8, the orifice 64 is frustoconical, so that a diameter D60, measured at the junction between the orifice 64 and the proximal surface 60 of the proximal dome cap 56, is greater than a diameter D62, measured at the junction between orifice 64 and the bearing surface 62.

The distal dome cap 57 comprises a distal surface 61. The distal surface 61 of the distal dome cap 57 has also a hemispherical shape, centered on the connector axis X43 and having a shape matching the recess 30. The distal dome cap 57 further comprises a bearing surface 63. The bearing surface 63 is parallel to and opposite the bearing surface 62 of the proximal dome cap 56 along the connector axis X43. The bearing surface 63 is located between the bearing surface 62 and the distal surface 61 along the connector axis X43.

The distal dome cap 57 further comprises an orifice 65. The orifice 65 passes through the distal dome cap 57 along the connector axis X43 and connects the distal surface 61 of the distal dome cap 57 to the bearing surface 63. A diameter of the orifice 65 is greater than or equal to a diameter of the extension 42. Advantageously, and as shown in the inserts of FIGS. 7 and 8, the orifice 65 is frustoconical, so that a diameter D61, measured at the junction between the orifice 65 and the distal surface 61 of the distal dome cap 57, is greater than a diameter D63, measured at the junction between orifice 65 and bearing surface 63.

Advantageously, and as shown in FIGS. 7 and 8, the proximal 56 and distal 57 caps are symmetrical with respect to the median plane P43, more particularly, the proximal 60 and distal 61 surfaces have an identical radius of curvature.

The ring 58 has a tubular shape, extending along a ring axis Y58, orthogonal to the connector axis X43 and belonging to the median plane P43. The ring 58 is thus symmetrical with respect to the median plane P43. The ring 58 is fixedly connected to the proximal 56 and distal 57 caps and is suitable for surrounding the vertebral rod 2.

When the connector 43 is threaded onto the extension 42, the proximal surface 61 of the distal dome cap 57 fits in with the recess 30 in a sphere-sphere contact. The sphere-sphere contact between the distal dome cap 57 and the recess 30, as well as the frustoconical orifices 64 and 65, allows the connector 43 to travel around the extension 42 and to take a plurality of orientations with respect to the plate 10. For example, the connector axis 43 can be at an angle, comprised between 0 and 15 degrees, to the extension axis X42, the ring axis Y58 can be at an angle, comprised between 0 and 15 degrees, to plate plane P10 and the connector can be rotated about the extension axis X42 at an angle comprised between 0 to 15 degrees. The connector 43 is thereby a polyaxial connector. The positions of the connector 43 corresponding to the maximum angle between connector axis X43 and the extension axis X42 are shown in dotted lines on insert a) of FIG. 8. The positions of the connector 43 and of the vertebral rod 2 corresponding to the maximum angle between the ring axis Y58 and the plate plane P10 are shown in dotted lines on insert b) of FIG. 8.

The connection device 15 is designed to move between a free configuration and a tight configuration. In the free configuration, shown in insert a) of FIG. 7 and inserts a) and b) of FIG. 8, the connector is threaded onto the extension 42, a slot 68 separates the bearing surfaces 62 and 63 and also splits ring 58. The vertebral rod 2 can then be easily inserted into the ring 58. The connector 43 is free to move between the different orientations described hereinabove.

The nut 44 comprises a tapped tightening part 70 so as to be screwed onto the extension 42, and a distal surface 71 with a shape matching the proximal surface 60. In the tightened configuration, shown in insert b) of FIG. 7, the nut 44 is screwed onto extension 42 and exerts a compressive force on connector 43. The distal surface 71 of the nut 44 then comes to bear against the proximal surface 60 of the proximal dome cap 56 in a sphere-sphere contact. The distal surface 61 of the distal dome cap 57 bears against the recess 30 also in sphere-sphere contact and the bearing surfaces 62 and 63 bear against each other along the connector axis X43. The slot 68 has a zero height measured along the connector axis X43. The proximal 56 and distal 57 caps are then co-radial, i.e. the proximal 60 and distal 61 surfaces form part of the same sphere. The ring 58 is tightened around the vertebral rod 2, so that the ring 58 cannot slide along the vertebral rod 2. Thereby, in the tightened configuration, the sphere-sphere contacts between, on the one hand, the nut 44 and the proximal surface 60, and, on the other hand, the recess 30 and the distal surface 61, allow the connector 43 to maintain an orientation among the orientations described hereinabove, without the connector being able to move and change orientation, e.g. due to a mechanical play. Thereby, the nut 44 fixes the orientation of the connector 43 but does not define said orientation.

The polyaxiality of the connector 43 facilitates the positioning of the ring 58 with respect to the vertebral rod 2, so that the vertebral rod 2 is not constrained by the positioning of the connector 43, but rather the connector 43 is apt to adapt to the position and to the orientation of the vertebral rod 2. The above serves in particular to limit the stresses exerted on the vertebral rod 2 and on the system 1 once the connection device 15 is in the tightened configuration.

Advantageously, the nut 44 comprises a screwing portion (not shown) supported by the tightening portion 70 and designed to detach from the tightening portion 70 when a torque equal to a maximum screwing torque is applied to the screwing portion. Thereby, the screwing torque applied to the nut 44 is constant, and the compression force applied to the connector 43 is constant. In particular, the above allows a person responsible for tightening to make sure that the nut 44 is correctly tightened, and that the connector 43 is in the tightened configuration, thereby limiting the risks of variations in the tightening torque from one system 1 to another. More particularly, when the system 1 is in the assembled configuration, the connection device 15 is in the tightened configuration and the nut 44 is screwed onto the extension 42 at a torque equal to the maximum screwing torque.

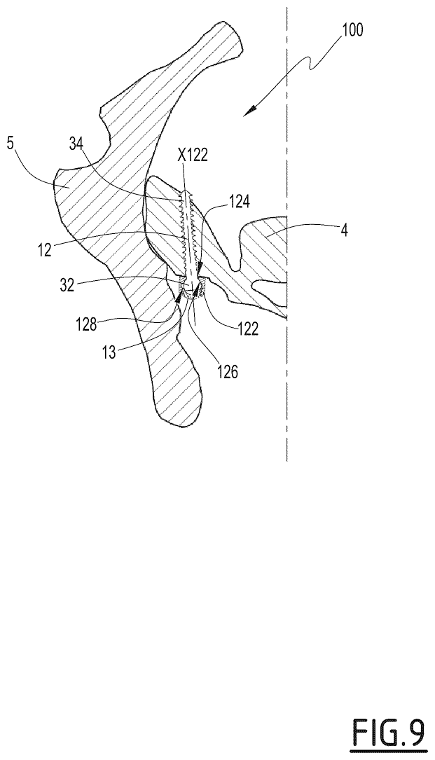

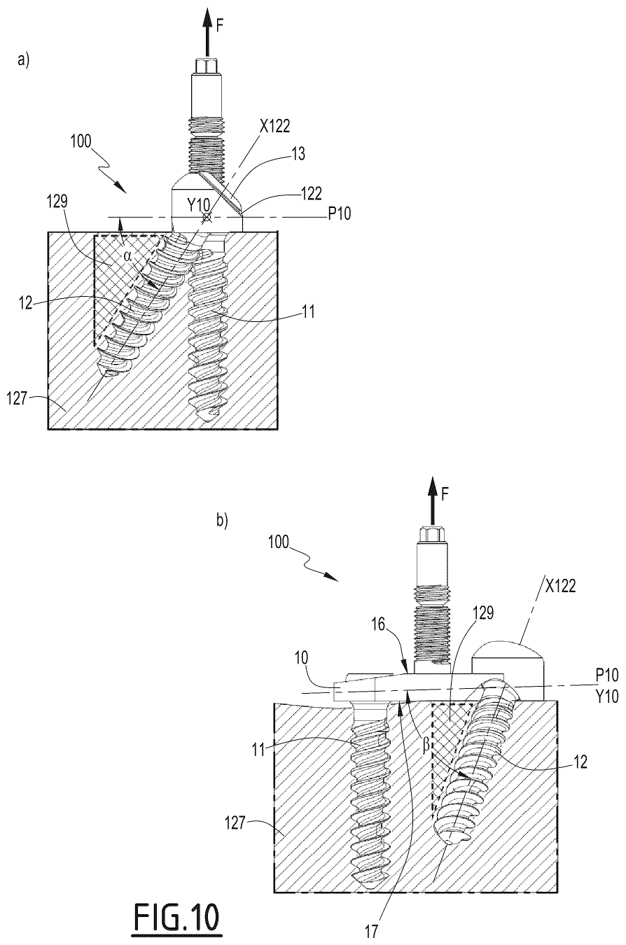

FIG. 9 and inserts a) and b) of FIG. 10 show a system 100 as an alternative embodiment of the system 1. The elements of the system 100 identical to the system 1 are referred to by the same reference signs and are not described again. The connector 43 and the nut 44 are not shown.

The system 100 differs from the system 1 by the second housing 122 thereof which replaces the second housing 22. The second housing 122 extends along a second axis X122. The second axis X122 and the plate axis Y10 form a second angle β, the second angle β being measured on the side of the plate 10 intended to be turned upwards with respect to the patient. In other words, the second angle β is formed in a geometric plane, formed by the plate axis Y10 and the second axis X122. In the embodiment considered in FIGS. 9 and 10, the second angle β is preferentially comprised between 45 and 70 degrees, preferentially between 55 and 65 degrees, or even equal to 60 degrees.

Taking into account the foregoing for the first and second embodiments described hitherto, it should thus be understood that the invention more generally covers values of the second angle β comprised between 45 and 110 degrees.

As can be seen in FIG. 9, the second housing 122 comprises a second hole 124, a second bottom 126 and a tapped surface 128. Apart from the value of the second angle β of the system 100, which differs from the value of the second angle β of the system 1, the second hole 124, the second bottom 126 and the tapped surface 128 are functionally or even structurally similar to the second hole 24, to the second bottom 26 and to the tapped surface 28, respectively, of the system 1.

Thereby, when the system 100 is in the assembled configuration, the second screw 12 extends lengthwise substantially along the second axis X122, i.e. the central longitudinal axis thereof coincides with the second axis X122, as illustrated schematically in FIG. 9 and the two inserts a) and b) in FIG. 10, or else forms with the latter an angle of only a few degrees, typically less than 15 degrees. In any case, the angulation of the second axis X122, as defined above through the first and second angles α and β, allows, in assembled configuration, the second screw 12 to penetrate the vertebra S2 of the sacrum 4, toward the promontory of the sacrum 4, until same can, once the second screw 12 is dimensioned in length in an ad hoc manner, pass through the vertebra S2, without reaching the iliac bone 5. The second screw 12 is advantageously transfixing, i.e. same passes through the vertebra S2 right through, more particularly the anterior and posterior corticals of the vertebra S2 of the patient's sacrum 4.

Similarly to what has been described for the system 1, when the system 100 is in service, same is subjected to the tear-off force F.

To better illustrate such phenomenon, inserts a) and b) of FIG. 10 show the first and second screws 11 and 12 penetrating into the bone material 127, the bone material 127 being represented schematically by a hatched zone representing the sacrum 4 of the patient. The bone material 127 comprises a bone region 129, shown by dotted lines and doubly hatched. The bone region 129 is located between the second screw 12 and the plate plane P10 and limits the movement of the second screw 12 due to the tear-off force F, which prevents the system 100 from being torn off. The existence of the region 129 is due to the angulation of X122, and in particular the value of the first angle α and, herein, of the second angle β. By means of the angulation of the second angle β, herein equal to 60 degrees, the bone region 129 opposing the movement of the second screw 12, has a volume greater than the volume of the bone region 29, ensuring good mechanical strength of the system 100. Such better mechanical strength, provided by the greater volume of the bone region 129 compared with the volume of the bone region 29, may partially or totally compensate for a lesser mechanical strength of the system 100 due to the fact that the second screw 12 does not penetrate into the iliac bone 5, contrary to what is provided for the system 1.

The system 100 is advantageous for patients for whom it is not possible to penetrate the iliac bone 5, providing a good mechanical strength without requiring to be fastened to both the vertebra S2 of the sacrum 4 of the patient and to the iliac bone 5 of the patient. Such is the case e.g. for pregnant women, or women who could become pregnant while wearing the system 100.

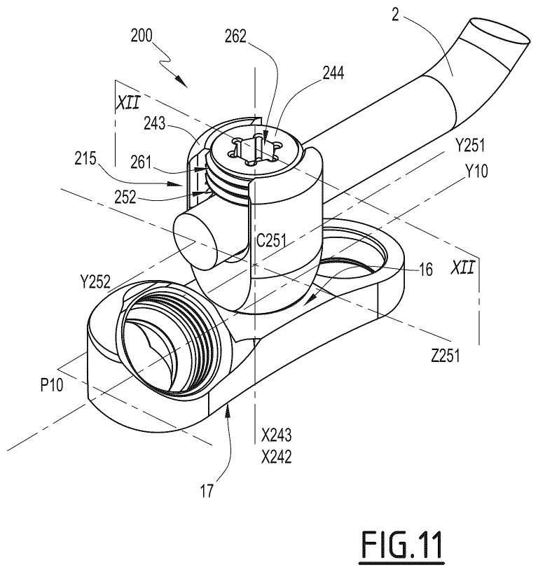

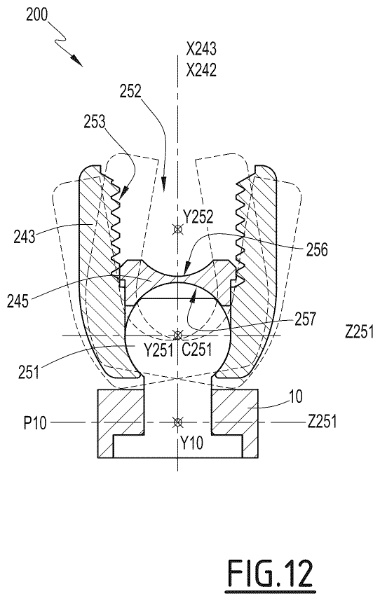

FIGS. 11 and 12 show a system 200 according to an alternative embodiment of the system 1. The elements of the system 200 identical to the system 1 are referred to by the same reference signs and are not described again. The first and second screws 11 and 12 and the locking cap 13 are not shown.

The 200 system differs from the system 1 in that same comprises a connection device 215 which replaces the connection device 15 of the 1 system. The connection device is located on the proximal face 16, between the two housings 21 and 22, and aligned with the plate axis Y10. The connection device 215 comprises a pin 242, a tulip-shaped piece 243, extending along a tulip-shaped piece axis X243, a cap 244 and a disc 245. The cap 244 is not shown in FIG. 12.

The pin 242, visible in FIG. 12, is fixedly supported by the plate 10, such that the plate axis Y10 passes through the pin 242. The pin 242 is e.g. welded to the plate 10. The pin 242 extends along the actuator axis pin X242. The pin 242 comprises a substantially spherical pin head 251 with a center C251.

The tulip-shaped piece 243 is articulated on the pin head 251, more particularly, the tulip-shaped piece 243 is in ball-and-socket linkage with the pin head 251, centered on the center C251. The tulip-shaped piece 243 can thereby rotate about the pin head 251 along the pin axis X242, along an axis Y251 passing through the center C251 and parallel to the plate axis Y10 and along an axis Z251, perpendicular to the pin axis X242 and to the plate axis Y10 and passing through the center C251, but cannot translate along these the axes X242, Y251 and Z251. The maximum pivoting of the tulip-shaped piece 243 about the axis Y251 is shown in dotted lines in FIG. 12, and the angle between the axis of the tulip-shaped piece X243 and the axis X242 is advantageously comprised between 0 and 15 degrees. Similarly, the angle between the axis of the tulip-shaped piece X243 and the axis X242, caused by the pivoting of the tulip-shaped piece 243 about the axis Z251, is advantageously comprised between 0 and 15 degrees. The tulip-shaped piece 243 is thus polyaxial.

The tulip-shaped piece 243 comprises a through opening 252 extending along an opening axis Y252 and comprising a tapped internal surface 253. The pin head 251 extends into the opening 252.

A disc 245 is arranged in the opening 252, on the pin head 251. The disc 245 comprises a proximal surface 256 matching the vertebral rod 2 and a distal surface 257 matching the pin head 251. Thereby, when the disc 245 is arranged in the opening 252, the distal surface 257 of the disc 245 bears against the pin head 251 and fits in with the shape of the pin head 251. The disc 245 is advantageously mounted tightly in the opening 252, so that same cannot fall out of the opening 252 and is rigidly attached to the tulip-shaped piece 243. In particular, the disc is suitable for pivoting about the pin head 251 at the same time as the tulip-shaped piece 243. Since the proximal surface 256 has a shape matching the vertebral rod 2, the proximal surface 256 is suitable for the vertebral rod 2 to be in contact with the proximal surface 256, fitting in with the shape of the vertebral rod 2. More particularly, the vertebral rod 2 can slide and pivot along the proximal surface 256. The above facilitates an optimal placement of the vertebral rod 2 in the tulip-shaped piece 243 during an assembly of the system 200.

The cap 244 comprises a threaded outer surface 261 so that same can be screwed into the opening 252. A recess 262 provided in the cap 244 facilitates the screwing of the cap 244 into the opening 252.

The connection device 215 can move between a free configuration and a tight configuration. In the free configuration, visible in FIG. 12, the tulip-shaped piece 243 is free to pivot according to the ball-and-socket movement about the pin head 251 and to take any orientation with respect to the axes X242, Y251 and Z251, described hereinabove. The vertebral rod 2 can slide and pivot in the opening 252 along the axis Y252.

When the connection device 215 is in the tightened configuration, as shown in FIG. 11, the cap 244 is screwed into the opening 252 and bears on the vertebral rod 2 and the disc 245. The vertebral rod 2 is thus bearing against the proximal surface 256 of the disc 245 and, due to the match between the vertebral rod 2 and the proximal surface 256, is held immobile in the opening 252. The distal surface 257 of the disc 245 also bears on the pin head 251 and, due to the match between the distal surfaces 257 and the pin head 251, forms a sphere-sphere contact, the disc 245 being held immobile with respect to the pin head 251. Thereby, the tulip-shaped piece 243 is held immobile with respect to the pin head 251. The orientations of the tulip part axis X243 and the opening axis Y252 are thereby fixed. In FIG. 11, the axes of the tulip-shaped piece X243 and of the pin X242 coincide, and the axes Y251 and Y252 are parallel.

The polyaxiality of the connection device 215, as for the connection device 15, facilitates the positioning of the tulip-shaped piece 243 with respect to the vertebral rod 2, so that the vertebral rod 2 is not constrained by the positioning of the tulip-shaped piece 243, but rather, the tulip-shaped piece 243 is apt to adapt to the position and to the orientation of the vertebral rod 2. The above serves in particular to limit the stresses exerted on the vertebral rod 2 and on the system 200 once the connection device 215 is in the tightened configuration.

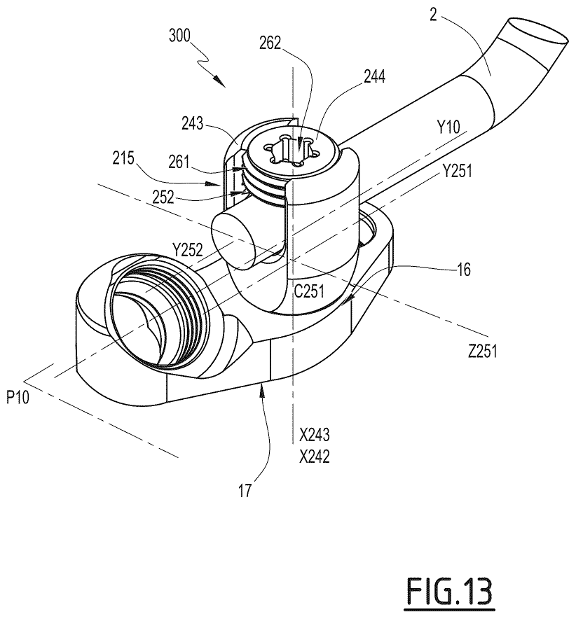

FIG. 13 shows a sacral fixation system 300, as an alternative embodiment of the system 200. The elements of the system 300 identical to the system 200 are referred to by the same reference signs and are not described again. The system 300 differs from the system 200 in that the connecting device 215 is not aligned with the plate axis Y10 but offset along a direction perpendicular to the plate axis Y10. Such location of the connection device 215 moves the connection device 215 away from the second housing 22 and thereby facilitates the insertion of the vertebral rod 2 and the screwing of the second screw 12 into the second housing 22, without risking having a contact between the vertebral rod 2 and the second screw 12 or the locking cap 13.

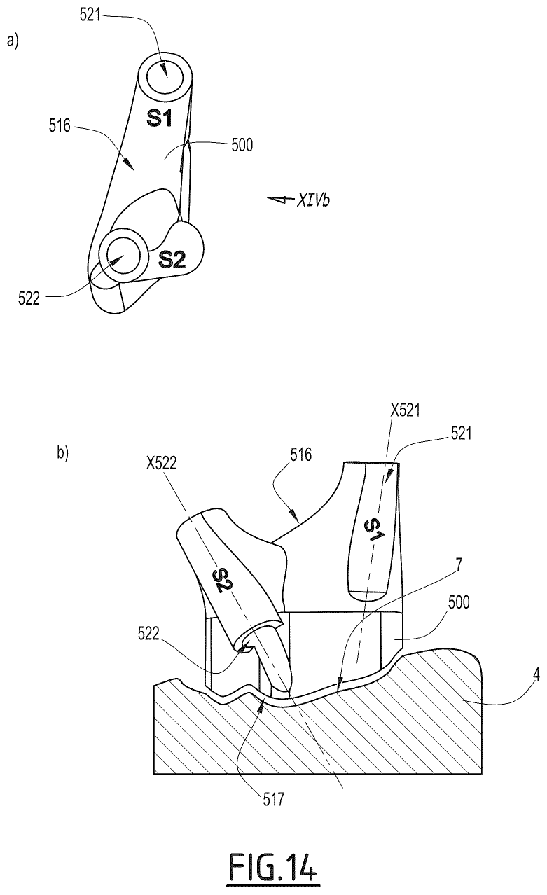

Inserts a) and b) of FIG. 14 represent a drill guide 500, also called a guide 500, belonging to a sacral fixation assembly also comprising one of the sacral fixation systems 1, 100, 200 or 300, e.g. the sacral fixation system 1. The guide 500 is used to drill two pre-holes in the patient's sacrum 4, in preparation for screwing the fastening plate 10 onto the patient's sacrum 4. The purpose of the drilled pre-holes is to facilitate the screwing of the first and second screws 11 and 12, by defining beforehand an orientation of the first and second screws 11 and 12.

The drill guide 500 comprises a main body 510 comprising a proximal surface 516 and a distal surface 517 opposite the proximal surface 516 in the sense that the proximal face 516 and the distal face 517 are separated from each other by the thickness of the main body 510, i.e. the smallest of the three dimensions of the main body 510. The distal surface 517 of the guide 500 is suitable for being brought into contact with the patient's sacrum 4, more particularly with a posterior region 7 of the patient's sacrum 4 and is morpho-adapted to the posterior region 7 of the patient, i.e. the distal surface 517 of the guide 500 matches the posterior region 7 of the patient. The guide 500 is suitable for being placed accurately so as to cover exactly the posterior region 7 of the patient.

A first guide hole 521 and a second guide hole 522 connect the proximal 516 and distal 517 surfaces of the guide 500 to each other. The first and the second guide holes 521 and 522 open out on the proximal and distal surfaces 516 and 517 of the guide 500 and extend respectively along a first guide axis X521 and a second guide axis X522. An orientation of the guide axes X521 and X522 is identical to the orientation of the first and second screws 11 and 12 when the 1 system is in the assembled configuration.

When the guide 500 is applied to the posterior region 7, a user of the guide 500, e.g. a surgeon, drills a first pre-hole, drilled through the first guide hole 521, along the guide axis X521. A second pre-hole, drilled through the second guide hole 522 is also drilled along the guide axis X522. Thereby, the pre-holes have an orientation identical to the orientation of the guide axes X521 and X522.

When the system 1 is in the assembled configuration, the first and second screws 11 and 12 are screwed into the first and second pre-hole, respectively. Thereby, the orientations of the first and second screws 11 and 12 are identical to the orientations of the first and second guide axes X521 and X522, respectively.

The aforementioned embodiments and variants can be combined with each other so as to generate new embodiments of the invention.

1. A sacral fixation system, comprising:
a plate comprising:
a proximal face and a distal face opposite the proximal face, a plate plane extending between the distal and proximal faces,
a first housing extending along a first axis by connecting the distal and proximal faces to each other, the first housing including both a first hole, which opens onto the distal face, and a first bottom, wherein the first hole is drilled, and which connects the first hole and the proximal face,
a second housing extending along a second axis by connecting the distal and proximal faces to each other, the second housing including both a second hole, which opens onto the distal face, a tapped surface, which opens onto the proximal face, and a second bottom, which has a concave shape, wherein the second hole is drilled, and which connects the second hole and the threaded surface, and
a plate axis belonging to the plate plane and intersecting with the first and second axes, the second axis forming, in projection onto a plane perpendicular to the plate axis, a first angle comprised between 30 and 60 degrees with the plate plane, and the second axis and the plate axis forming a second angle comprised between 45 degrees and 110 degrees;
a first screw, which, when the system is in an assembled configuration, is received in the first housing, extending into the first hole and emerging from the distal face so as to penetrate the vertebra S1 of the sacrum of a patient;
a second screw which comprises a threaded rod and a head, the head being substantially spherical matching the second bottom, the second screw being, when the system is in the assembled configuration, received in the second housing such that the head bears against the second bottom and the rod extends into the second hole and emerges from the distal face so as to be able to penetrate at least into the vertebra S2 of the sacrum of the patient;
a locking cap which comprises a skirt having an outer face threaded to match the tapped surface and an inner face of which matches the head of the second screw, and which, when the system is in the assembled configuration, is received in the second housing by interposing the skirt radially between the head of the second screw and the tapped surface, so that the head of the second screw is received in the skirt and is pressed against the inner face; and
a connecting device which, when the system is in the assembled configuration, is arranged on the proximal face between the first and second housings and connects the plate to a vertebral rod.

2. The system according to claim 1, wherein the second angle is comprised between 70 degrees and 110 degrees, preferably comprised between 85 and 95 degrees, and the second screw is dimensioned, in the assembled configuration, to pass through the vertebra S2 and reach the iliac bone of the patient.

3. The system according to claim 1, wherein the plate further comprises a recess and the connecting device comprises:
a threaded extension fixedly connected to the recess of the plate, the extension extending along an extension axis;
a connector, comprising a proximal dome cap and a distal dome cap, the distal dome cap matching the recess, each dome cap comprising:
an orifice with a diameter greater than a diameter of the extension, the orifices of the proximal and distal dome caps being coaxial and centered on a connector axis,
a bearing surface, the bearing surfaces facing each other along the connector axis;
each dome cap being movable along the connector axis in order to bring the bearing surfaces into contact with each other along the connector axis and to tighten the connector around the spinal rod,
a nut, including a tightening portion, the tightening portion comprising a distal surface matching the proximal dome cap,
and wherein, when the system is in the assembled configuration:
the connector is fitted onto the extension through the ports of the distal and proximal dome caps; and
the nut is screwed onto the extension, the distal surface bears against the proximal dome cap, the nut holds the distal dome cap against the recess and holds the bearing surfaces in contact along the connector axis; and
the connector is tightened around the vertebral rod.

4. The system according to claim 3, wherein the proximal and distal dome caps are hemispherical and when the system is in the assembled configuration, the lower and upper dome caps are co-radial.

5. The system according to claim 3, wherein the nut further comprises a screwing portion supported by the tightening portion, configured to detach from the tightening portion when a torque equal to a maximum screwing torque is applied to the screwing portion.

6. The system according to claim 3, wherein the extension includes a line of least resistance making same breakable when a bending or torsional torque equal to a bending or torsional breaking torque is applied to the extension.

7. The system according to claim 1, wherein the connection device comprises:
a pin, fixedly supported by the plate;
a tulip-shaped piece extending along a tulip-shaped piece axis, the pin and the tulip-shaped piece forming therebetween, a ball-and-socket linkage, the tulip-shaped piece comprising a threaded through opening;
a disc, comprising a distal surface matching the pin and a proximal surface matching the vertebral rod;
a cap, suitable for being screwed into the opening of the tulip-shaped piece, when the system is in the assembled configuration:
the disc is arranged in the opening of the tulip-shaped piece, the distal surface of the disc bearing against the pin;
the vertebral rod passes through the opening of the tulip-shaped piece, bearing against the proximal surface of the disc;
the cap is screwed into the opening of the tulip-shaped piece and holds the vertebral rod, the disc and the pin bearing against each other.

8. The system according to claim 7, wherein the plate axis passes through the pin.

9. The system according to claim 7, wherein the pin is offset from the plate axis along a direction perpendicular to the plate axis.

10. The system according to claim 1, wherein the second angle is comprised between 85 and 95 degrees, and the second screw is dimensioned, in the assembled configuration, to pass through the vertebra S2 and reach the iliac bone of the patient.

11. A sacral fixation assembly, comprising the system according to claim 1 and a drill guide comprising:
a distal surface morpho-adapted to a posterior region of the patient's sacrum;
a first guide hole centered along a first guide axis, and
a second guide hole centered along a second guide axis, the first guide hole and the second guide hole being oriented identically to the first and the second screws when the system is in the assembled configuration.
